(19) United States
(12) Patent Application Publication
(10) Pub. No.: US 2024/0299898 A1
(43) Pub. Date: Sep. 12, 2024

(54) Method to Aerosolize Nanoparticle Formulations

(71) Applicant: Medspray B.V., Enschede (NL)

(72) Inventor: Cornelis Johannes Maria Van Rijn, Amsterdam (NL)

(21) Appl. No.: 18/269,012

(22) PCT Filed: Dec. 24, 2021
(86) PCT No.: PCT/IB2021/062306

§ 371 Date: Jun. 22, 2023

(30) Foreign Application Priority Data
Dec. 24, 2020 (NL) 2027229

(51) Int. Cl. B01J 13/00

(52) U.S. Cl. CPC B01J 13/0095

(57) ABSTRACT

For delivering nanoparticles in an atmosphere, a liquid formulation that comprises said nano-particles is provided and pressurized to an elevated operating pressure p. Said liquid formulation is fed at said elevated operating pressure through a spray nozzle orifice of at leastone spray orifice to discharge said liquid formulation as a jet of consecutive liquid droplets that contain at least one nano-particle of said nanoparticles. Said nanoparticles have a length λ and a maximum length λₘₐₓ before breakage upon elongation and said liquid formulation is subjected to a wall shear rate γ_{wall} [per second] while passing through said spray nozzle orifice. According to the invention said liquid formulation is exposed within said spray nozzle orifice to said wall shear rate during a limited shear time t that is less than λₘₐₓ/(Δγ_{wall}) seconds.

The present invention relates to a method to aerosolize nanoparticle formulations, including formulations containing proteins, peptides, DNA, RNA, vesicles, liposomes and antibodies.

Current aqueous aerosolization devices are for instance nebulizers and soft mist inhalers. Nebulizers generate aerosols by using either compressed air, an ultrasonic power source or a vibrating mesh/membrane to disperse the formulation. Soft mist inhalers produce a mist of fine aqueous droplets by collision of microjets at a very high velocity.

European patent application EP 3.563.894 discloses an example of such a soft mist inhaler, being used to nebulize a non-Newtonian fluid, like in particular a suspension or emulsion or a liposomal fluid that uses nozzles with a small diameter. This known nebulizer comprises a fluid pump for pressurizing the fluid to an operational pressure of 5 to 250 Mpa and forcing the pressurized fluid through two mutually inclined flow channels having a hydraulic diameter in the range of 3 to 20 microns. This produces two crossing fluid jets at extreme high velocity that will impinge on one another to produce a mist of fine aqueous droplets. This collision of both fluid jets at high velocity has a significant impact on the fluid and any particles contained therein. It turns out that fragile, delicate nano-particles tend to be damaged under these conditions and in many cases are unlikely to survive the aerolization process.

International patent application WO 02/13786 A2 discloses a device and method of producing particles out of a stream of a fluid in which a vibrator is used to force the fluid stream into individual droplets. The fluid is being charged electrically and the vibrator comprises particularly an ultrasonic piezoelectric transducer that is able to act on the charged fluid emerging from a nozzle channel having a diameter of at least 100 micron. This known method and device subjects the fluid to considerable mechanical stress due to the ultrasonic source or vibrating mesh/membrane. Molecules contained in the fluid are exposed to that stress causing shear stress induced degradation of these molecules. Any delicate and fragile molecules are, hence, prone to degradation using this known device and method. Moreover, this spraying mechanism together with the nozzle diameter of at least 100 micron renders this known device only suitable for applications at relative large dimensions. Decreasing the nozzle diameter from 100 micron to 5 micron, for instance, would increase the damage done to any encapsulated compound by factors of magnitude. This known method and device, hence, have no potential use in a sub-micron range requiring smaller nozzles and causing even more damage to any compounds contained by the fluid due to increasingly large wall shear forces when reducing the nozzle diameter.

It is inter alia an object of the present invention to provide a method of aerolizing a fluid composition that contains nano-particles that maintains the molecular integrity when aerosolizing stress-sensitive nanoparticles, such as formulations containing complex proteins, peptides, long chain DNA & RNA, large vesicles, liposomes and/or antibodies.

To that end the present invention provides a method for delivering nanoparticles in an atmosphere, comprising:
providing nano-particles having a particle length (A) in a liquid to form a liquid formulation;
pressurizing said liquid formulation to a moderate operating pressure (p) to provide a pressurized liquid formulation; and
feeding said pressurized liquid formulation through a spray nozzle orifice, having a channel length (L) between an inlet and an outlet of said orifice and an average channel diameter (H) between said inlet and said outlet, to create a liquid stream of said liquid formulation with a velocity;
   wherein said moderate operating pressure is held below 10 Mpa and said velocity less than 100 m/s;
   wherein said orifice has a channel length (L) that is shorter than said average channel diameter (H); and
   wherein said liquid stream is collected at said outlet as a jet of consecutive liquid droplets that each contain at least one nanoparticle of said nanoparticles.

In a specific embodiment the method according to the invention is characterized in that said liquid formulation comprises shear stress-sensitive nano-particles taken from a group, containing complex proteins, large biological molecules, long chain DNA & RNA, viruses, large vesicles, liposomes, bacteriophages, and antibodies, wherein said orifice has a channel length (L) that is shorter than half said average channel diameter (H), and preferably wherein said orifice has a channel length (L) that is at most a quarter of said average channel diameter (H). In practice it turns out that the integrity of even these delicate and fragile molecules and nano-particles may be preserved when the preceding conditions are fulfilled.

In an embodiment of the method according to the invention said liquid formulation comprises protein and/or antibody molecules, particularly nucleotide compounds like DNA or RNA molecules, with a molecular weight that is larger than 100.000 g/mol.

In a further embodiment of the method according to the invention said liquid formulation comprises bacteriophages with an average size larger than 20 nanometre.

In a further embodiment of the method according to the invention said liquid formulation comprises lipid nanoparticles or liposomes, in particular lung surfactants, of which said length is larger than 20 nanometre, wherein particularly said liquid formulation comprises vesicles that have a content comprising nanoparticles taken from a group, containing proteins, biological molecules, DNA, RNA, vaccines, viruses, bacteriophages and antibodies with a molecular weight above 100.000 Da.

In a further embodiment of the method according to the invention said nozzle orifice has a substantially constant diameter (H) that is between 1 micron and 10 micron.

In a further embodiment of the method according to the invention said nozzle orifice has an average diameter (H) between 1 micron and 10 micron; and wherein said orifice tapers over at least part of said length from said inlet to said outlet, wherein particularly said nozzle orifice is provided with a positive taper, narrowing from said inlet entrance to said outlet at substantially a tapering between 5° and 45°.

In a further embodiment of the method according to the invention an inner wall of said nozzle orifice is provided with a hydrophobic slip flow enabling coating.

In a particular embodiment the method according to the invention is characterized in that said nanoparticles have a maximum particle length λₘₐₓ before breakage upon elongation; wherein said liquid formulation is subjected to a wall shear rate γ_{wall} [per second] while passing through said spray nozzle orifice; and wherein said liquid formulation is exposed within said spray nozzle orifice to said wall shear rate during a shear time (t) that is less than λₘₐₓ/(λ·γ_{wall}) seconds.

For lipid nanoparticles, vesicles, liposomes and other spherical core-shell particles is the ratio λₘₐₓ/λ experimentally found to be in a typical small range between 3-5, and on average 4 nearly independent of the particle size above a size of 20 nm. For linear, coiled and stiff molecules or nanoparticles such as DNA, RNA, charged polyelectrolytes, etc is the ratio λₘₐₓ/λ experimentally found to be in a range between 4 and 6 for molecules with a molecular or nanao-particle weight above 100.000.

Short molecules exhibit a less maximum elongation, but on the other hand are also more stiff and less susceptible to rupture due to the shear forces. Larger molecules (above 500.000 Da) exhibit a larger maximum elongation, but on the other hand are more susceptible to rupture due to the shear forces. These fluid mechanic effects tend to compensate each other for the susceptibility for rupture.

For flexible molecules such as proteins, and large antibodies is the ratio λₘₐₓ/λ experimentally found to be in a typical range between 2 and 5 for molecules with a molecular weight above 100.000. For semi-rigid nanoparticles such as bacteriophages and viruses, the ratio λₘₐₓ/λ is found experimentally to be in a broad range between 2 and 6 for a nano-particle weight above 100.000. To be on the safe side, a preferred embodiment of the method according to the invention is characterized in that the ratio λₘₐₓ/λ is set at 4.

For extremely stress-sensitive, highly complex and large nanoparticles (>100 nm) such as e.g. pulmonary surfactants is the ratio λₘₐₓ/λ experimentally found to be in a typical range between 2 and 6. Also here the said nanoparticle size effects tend to compensate each other with respect to the susceptibility for rupture. However for these highly complex type of nanoparticles, including the compensation effect, the ratio λₘₐₓ/λ can best be set at 2 instead of 4.

The volume averaged degradation is determined by γₚₐᵣₜ with a value always smaller than the wall shear rate γ_{wall}. This means that according to this embodiment the general criterium to avoid degradation of the nanoparticle formulation, i.e. γ_{wall} t=λₘₐₓ/λ<4 is set on the safe side and will protect stress-sensitive nanoparticles with a size λ above 20 nm and/or a molecular weight above 100.000 Da. For highly complex and large nanoparticles (λ>100 nm) a more safer margin can be set at γ_{wall} t=λₘₐₓ/λ<2.

With this mutual condition of wall shear rate and shear time it appears surprisingly possible to maintain the molecular integrity of both long chain molecules and vesicles during the aerosolization process to make droplets between 2 and 20 micron using a spray nozzle having a small diameter between 1 and 10 micron and a length not exceeding 2 micron. Surprisingly it has been found that even by using large wall shear rates, at least ten to hundred times higher than in conventional nebulizers, during a very short time molecular integrity of the nanoparticle formulation may still be preserved.

It is an insight according to the invention that these conditions are particularly fulfilled by using a spray orifice with a small diameter between 1 and 10 micron and a nozzle length less than 2 micron. Thereby the nozzle is constructed in such a way that even despite a high wall shear rate γ_{wall}, the nanoparticles in the fluid further away from the nozzle wall suffer less from the high wall shear rate γ_{wall}. The wall shear rate γ_{wall} is the velocity difference near the wall and perpendicular to the wall divided by the distance to the wall with dimension per second. Shear stress on the nano-particle originates from local velocity gradients over the length of the particle, and the velocity difference over the nanoparticle divided by the size of the nanoparticle, is here named the particle shear rate yₚₐᵣₜ with dimension per second.

It is a further insight behind the invention that the drag force exerted on the nanoparticle, that may induce tensioning and subsequent breakage of the nanoparticle, is mainly proportional to the particle shear rate γₚₐᵣₜ, with a value that is substantially less than the wall shear rate γ_{wall}. In a long pipe with a parabolic velocity distribution the wall shear rate γ_{wall} and particle shear rate γₚₐᵣₜ near the wall is 8V/H. However the particle shear rate γₚₐᵣₜ in the middle of the pipe is zero. Moreover, the liquid flows in the middle of the pipe with velocity 2V, whereas there is substantially no flow near the wall of the pipe. The volume averaged particle shear rate γₚₐᵣₜ in a pipe, depending on the type of flow and the length of the pipe, can therefore vary between about 4V/H, for a long or medium pipe, up to a substantially smaller value, for a short pipe or orifice. In order to reduce the volume averaged particle shear rate γₚₐᵣₜ, according to the invention a relatively short orifice, compared to its diameter, is used as it appears more favourable to use relatively short pipes or orifices.

It is an insight according to the invention that large macromolecules or large vesicles have not sufficient rigidity or intrinsic resistance to withstand deformation forces due to hydrodynamic shear forces proportional to the particle shear rate γₚₐᵣₜ. A further insight underlying the invention is that the volume averaged particle shear rate γₚₐᵣₜ is substantially less than the wall shear rate γ_{wall} near the wall, and that a safe condition to maintain the integrity of the nanoparticles is that the shear time is preferably less than λₘₐₓ/(λ·γ_{wall}). In a shearing time of λₘₐₓ/(λ·γ_{wall}) seconds the nanoparticle near the wall is then elongated or deformed with a length λ about γ_{wall} times λ times λₘₐₓ/(λ·γ_{wall})=λₘₐₓ. The aerosolization device should be capable in providing the conditions to impose a wall shear rate γ_{wall} per second during an ultra-short shear time t of less than λₘₐₓ/(λ·γ_{wall}) seconds. As a result not only the nanoparticles near the nozzle wall will preserve their integrity, but also the nanoparticles more remote from the nozzle wall.

In a further embodiment of the method according to the invention said wall shear rate γ_{wall} IS well above 100.000, in particular above 1.000.000 per second.

In a further embodiment of the method according to the invention said nano-particles comprises macromolecules with a molecular weight that is larger than 100.000 g/mol; and wherein said macromolecules or nano-particles have a ratio λₘₐₓ/λ of at least 2, and preferably a ratio λₘₐₓ/λ of at least 4.

In a further embodiment of the method according to the invention the nozzle orifice produces a jet with an average velocity V coming out of an orifice with a diameter H and a length Land shearing conditions:

$${Υ}_{part} < {Υ}_{wall} = 8 V / H and t = L / V = {λ}_{max} / ( λ \cdot {Υ}_{wall} ) < {λ}_{max} / ( λ \cdot {Υ}_{part} ) \\ or \\ {Υ}_{part} \cdot t < {Υ}_{wall} \cdot t = 8 L / H .$$

A particular embodiment of the method according to the invention is characterized in that the nanoparticle formulation during aerosolization, producing aerosol droplets with a size of 2-20 micron, through a small nozzle with a diameter H between 1 and 10 micron and a length L smaller than 2 microns, is subject to a wall shear rate γ_{wall} substantially above 1.000.000 per second during an ultra-short shear time t substantially less than 4×10-6 sec (with a maximum nanoparticle elongation λₘₐₓ/λ of 4 we get γ_{wall}·t<4) The condition γ_{wall}·t=8L/H gives then that L<H/2, or the nozzle length L should not exceed half the diameter H of the nozzle, when aerosolizing liquid formulations comprising shear stress-sensitive nanoparticles, such as proteins, peptides, DNA & RNA, vesicles, liposomes, lipid nanoparticles and antibodies.

A further reduction of the nozzle length will reduce the total nozzle wall surface and hence its contribution to the wall shear on the nanoparticles and with preference the nozzle length L should not exceed a quarter of the nozzle diameter, thus L<H/4. This applies specifically for highly complex and stress-sensitive nanoparticles having a size typically larger than 100 nm. With preference, according to the invention, nozzles are used with orifices having a short length as they exhibit plug and slip flow conditions that diminish the nanoparticle shear rate during operation. In a plug flow condition the particle shear rate away from the nozzle wall is considerably less due to a more even velocity distribution and creating a slip flow condition (e.g. using a hydrophobic nozzle wall), diminishes also the particle shear rate near the nozzle wall. With preference nozzles do combine both plug and slip flow at the same time herewith diminishing the volume averaged particle shear rate.

A further embodiment of the method according to the invention is characterized in that the device produces a jet with an average velocity V coming out of nozzle with a diameter H between 1 and 10 micron and a length L, such that L<H/2, and that the nozzle pressure is between 10 and 100 bar. Increasing the velocity V of the jet above 10 m/s by increasing the nozzle pressure has also been found to diminish the amount of degradation and an optimum operational pressure of about 30-80 bar has been found to diminish the risk of degradation of the nanoparticle formulation.

Average flow velocities of about V=100 m/s above at operating pressure of 100 bar or above have been found detrimental for the nanoparticle integrity possibly because of the high operational pressure beyond 100 bar to achieve such velocities that may cause severe turbulence in the formulation disrupting the nanoparticles. Also at such high nozzle pressures the liquid jet coming out of the nozzle entails so much kinetic energy, that the jet immediately disintegrates before it can produce a jet of consecutive droplets that are about twice the diameter of the jet. The unwanted turbulence and disintegration of the jet also lead to a strong increase of the volume averaged particle shear rate and increased levels of disruption of the nano-particles. This unwanted transition to turbulence and disintegration may occur whenever the ratio of jet mass density p, times jet velocity V times nozzle diameter H divided by the jet viscosity n becomes larger than 2.500 (ρ·V·H/η>2500). A further preferred embodiment of the method according to the invention is, hence, characterized in that a product of a mass density (φ of said fluid, a fluid velocity (V) inside said orifice and said nozzle diameter (H) divided by a viscosity (n) of said fluid, expressed as ρ·V·H/η, is maintained below 2.500.

In a further preferred embodiment the method according to the invention is characterized in that said nozzle orifice is part of a collection of substantially identical nozzle orifices that extend through a common membrane layer that is supported by a substrate, wherein said substrate has at least one cavity extending to said nozzle orifices of said collection of orifices, and wherein said liquid formulation is delivered at said operating pressure jointly to said cavities to supply said nozzle orifices of said collection of orifices.

Satisfactory results have also been obtained with slightly positively or negatively tapering short orifices with a minimum diameter H between 1 and 10 micron and a total length L smaller than 2 microns with tapering angles between 5° and 45°.

### BRIEF DESCRIPTION OF THE DRAWINGS

FIG. 1 illustrates the breakup of a macromolecule in a shear flow.

FIG. 2 illustrates the breakup of a vesicle in a shear flow.

FIG. 3 illustrates the breakup of a vesicle in a strong shear flow.

FIG. 4 illustrates the passage of a vesicle through a long pipe.

FIG. 5 illustrates the passage of a vesicle through an orifice.

FIG. 6 illustrates the passage of a vesicle through a positively tapered orifice.

FIG. 7 illustrates the passage of a vesicle through a negatively tapered orifice.

### DESCRIPTION OF THE PREFERRED EMBODIMENTS

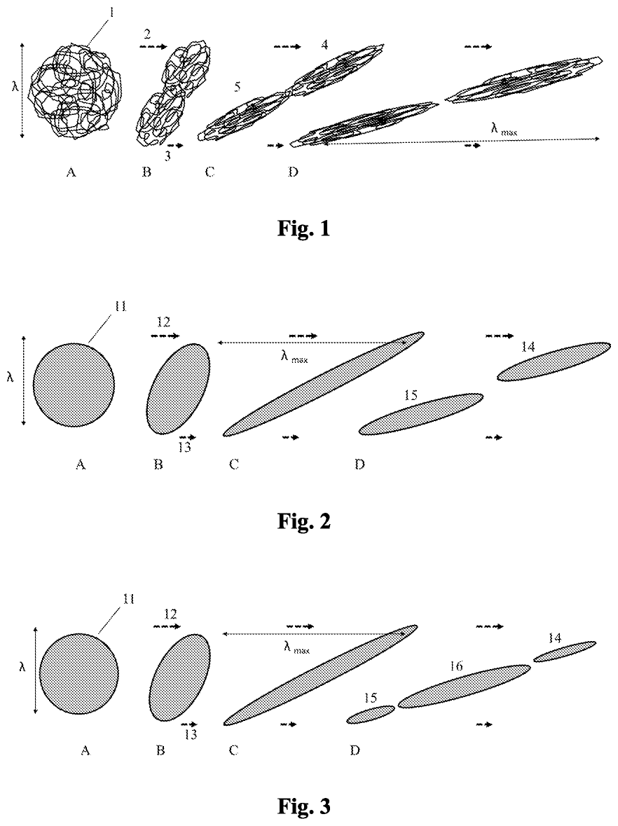

FIG. 1 illustrates the breakup of a macromolecule in a shear flow. FIG. 1A shows a normal globular macromolecule (1) with a gyration diameter λ without the presence of a shear flow. FIG. 1B shows the initial stretching of the globular macromolecule in the presence of a shear flow indicated by arrows (2,3). FIG. 1C shows the further stretching to a maximum length λₘₐₓ of the macromolecule and the upper part of the macromolecule moving with a higher velocity starts separating form the lower part with a smaller velocity. The upper part (4) and the lower part (5) are only separated by one or two remaining covalent bonds. FIG. 1D shows the macromolecule after breaking in two parts due to the hydrodynamic shear forces exerted on the upper and lower part of the macromolecule, that cause breaking of the covalent bonds in the middle of macromolecule.

Long and heavy macromolecules such as large proteins, long RNA and DNA chains, antibodies, etc. having a molecular weight above 100.000 g/mol are susceptible to shear induced degradation. In Table 1 below some results are summarized on shear degradation of immunoglobulins (protein/antibody) with varying molecular weight during aerosolization.

TABLE 1

| | | Survival (%) | | | | |
|---|---|---|---|---|---|---|
| | Molecular | Vibrating | Survival (%) | Survival (%) | Survival (%) | Survival (%) |
| Macromolecule | Weight | Membrane | t = 200 ns | t = 200 ns | t = 50 ns | t = 10 ns |
| (weight %) | (g/mol) | Nebulizer | Orifice 2 μm | 25° tapering | Orifice 2 μm | Orifice 2 μm |
| Bovine IGG (0.1%) | 160.000 | 85 | 100 | 100 | 100 | 100 |
| Bovine IGG (1%) | 160.000 | 63 | 100 | 100 | 100 | 100 |
| Bovine IGG (5%) | 160.000 | 33 | 98 | 99 | 99 | 94 |
| Bovine IGG (10%) | 160.000 | 20 | 97 | 98 | 97 | 93 |
| Bovine IGG (20%) | 160.000 | 15 | 96 | 97 | 98 | 93 |
| Bovine IGM (0.1%) | 900.000 | 23 | 100 | 100 | 100 | 80 |
| Bovine IGM (1%) | 900.000 | 18 | 100 | 100 | 100 | 82 |
| Bovine IGM (5%) | 900.000 | 8 | 98 | 99 | 99 | 73 |
| Bovine IGM (10%) | 900.000 | 5 | 97 | 100 | 100 | 70 |

Using a vibrating membrane nebulizer with a formulation reservoir of 1 ml aerosol droplets in the range of 2-10 micron have been produced during a nebulization time of 8 minutes. Diluted formulations with protein/antibodies having a larger molecular weight (>100.000 g/mol) clearly suffer from shear degradation. According to the invention aerosol droplets with an average size of 4 micron have also been made by passing the same formulations with the antibodies through an orifice having a diameter H=2 micron and a length L=1 micron at an average velocity V=5 m/s, and the passage time t is about 200 nanosecond. Due to this short shearing time according to the invention the macromolecules present in the droplets have suffered substantially less from degradation. Using an orifice with a 25° positively tapered profile the antibodies hardly suffer from degradation even for molecular weights well above 1.000.000 g/mol. At an average velocity of V=20 m/s the passage time t through an untapered orifice is about 50 nanosecond and less degradation is found than in the case of an average velocity of 5 m/s. Increasing the average velocity to V=100 m/s (t=10 ns) however leads to a substantial degradation of the formulation. Decreasing the nozzle length L to 0.5 micron (H=2 micron), gives a degradation loss that is relatively 15% less than in the case of a 1 micron length nozzle.

Macromolecules such as DNA and RNA nucleic acids are frequently used in gene therapy, in particular non-viral gene delivery vectors with messenger RNA (mRNA) and mini-DNA vectors. These macromolecules can range between 100-10.000 nucleotides or base pairs with corresponding contour lengths from 30 nanometre to 3 micron, making them susceptible to shear-induced degradation. In Table 2 below some results are summarized on shear degradation of a number of these vector molecules.

TABLE 2

| | | | Survival (%) | | | |
|---|---|---|---|---|---|---|
| | | Molecular | Vibrating | Survival (%) | Survival (%) | Survival (%) |
| Length in | Vector | Weight | Membrane | Orifice 2 um | Orifice 2 um | Orifice 2 um |
| Basepairs | Molecule | (g/mol) | Nebulizer | t = 200 ns | t = 50 ns | t = 10 ns |
| 336 | mv281 | 208.000 | 85 | 100 | 100 | 95 |
| 1109 | mv-KB4TAL-GLuc | 698.000 | 63 | 100 | 100 | 88 |
| 1714 | mv-CMV-mCherry | 1,080.000 | 30 | 99 | 99 | 84 |
| 3000 | pBLUESCRIPT | 1,890.000 | 10 | 98 | 99 | 76 |
| 5302 | pCR2.1-norE | 3,340.000 | 2 | 95 | 97 | 69 |

Using a vibrating membrane nebulizer with a formulation reservoir of 1 ml aerosol droplets in the range of 2-10 micron have been produced during a nebulization time of 8 minutes. Formulations with DNA or RNA vectors (5 μg/ml) having a larger molecular weight (>200.000 g/mol) clearly suffer from degradation. According to the invention aerosol droplets with an average size of 4 micron have also been made by passing the same formulations with the vector molecules through an orifice having a diameter H=2 micron and a length L=1 micron at an average velocity V=5 m/s. Because of plug flow conditions the volume averaged wall shear rate will be significantly lower than 4V/H=10×10⁶ per second, whereas the passage time t about 200 nanosecond. Due to this short shearing time according to the invention the vector molecules present in the droplets have suffered substantially less from degradation. At an average velocity of V=20 m/s the passage time t through an untapered orifice is about 50 nanosecond and less degradation is found than in the case of an average velocity of 5 m/s. Increasing the average velocity to V=100 m/s (t=10 ns) however leads to a substantial degradation of the formulation. Using an orifice with a 25° negatively tapered profile with a length of 1 micron the antibodies hardly suffer from degradation even for molecular weights well above 1.000.000 g/mol at velocity V=50 m/s. Decreasing the nozzle length L to 0.5 micron (H=2 micron, not tapered), gives a degradation loss that on average is relatively 10% less than in the case of a 1 micron length non-tapered nozzle. Results with nozzles having a hydrophobic fluor coating with a water contact angle of 112° also yield a lower degradation loss typically relatively 5-15% less compared to uncoated nozzles.

FIG. 2 illustrates the breakup of a vesicle or a liposome in a shear flow. FIG. 2A shows a spherical vesicle (11) with a size λ without the presence of a shear flow. FIG. 2B shows the initial stretching of the vesicle in the presence of a shear flow indicated by arrows (12,13). FIG. 2C shows further stretching to a maximum length λₘₐₓ of the vesicle, because the upper part of the vesicle is moving with a higher velocity than the lower part. FIG. 2D shows the vesicle after breaking in two parts (14,15) due to the hydrodynamic shear forces exerted on the upper and lower part of the vesicle, that cause breaking in the middle of the vesicle.

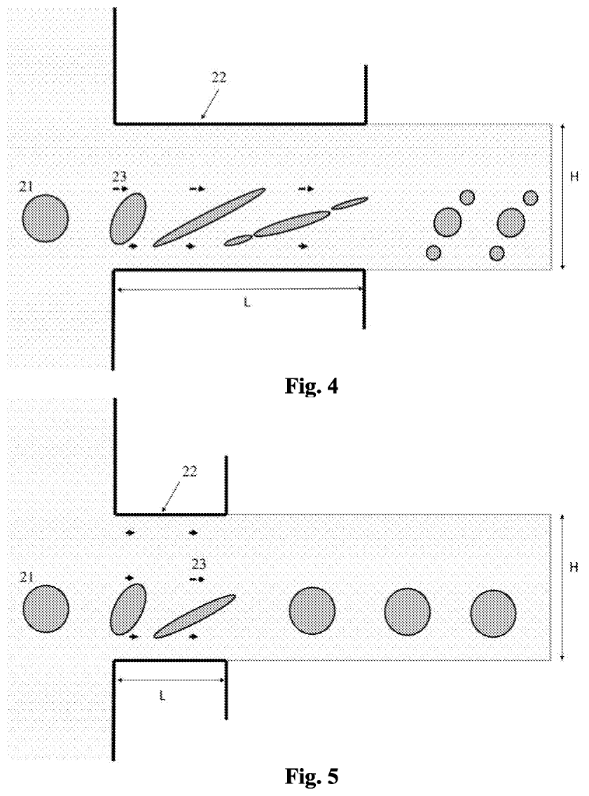

FIG. 3 illustrates the breakup of a vesicle or a liposome in a strong shear flow. FIG. 3A shows the vesicle (11) with a size λ without the presence of a shear flow. FIG. 3B shows the initial stretching of the vesicle in the presence of the shear flow indicated by arrows (12,13). FIG. 3C shows further extreme stretching of the vesicle to a maximum length λₘₐₓ, because the upper part of the vesicle is moving with a much higher velocity than the lower part. FIG. 3D shows the vesicle after breaking in three parts (14,15,16) due to the large hydrodynamic shear forces exerted on the vesicle, that cause breaking in the vesicle in three parts.

FIG. 4 illustrates the breakup of a vesicle or a liposome (21) in a long pipe (22) with length L and diameter H. Before the entrance of the pipe the vesicle (21) is round due to the absence of a large shear flow. When the vesicle enters the pipe, it will be subject to a shear flow (23), and after the stretching stages of the vesicle it breaks in three elongated parts before it exits the pipe. The three parts travel further in the jet and due to the absence of shear the three parts attain their spherical shape.

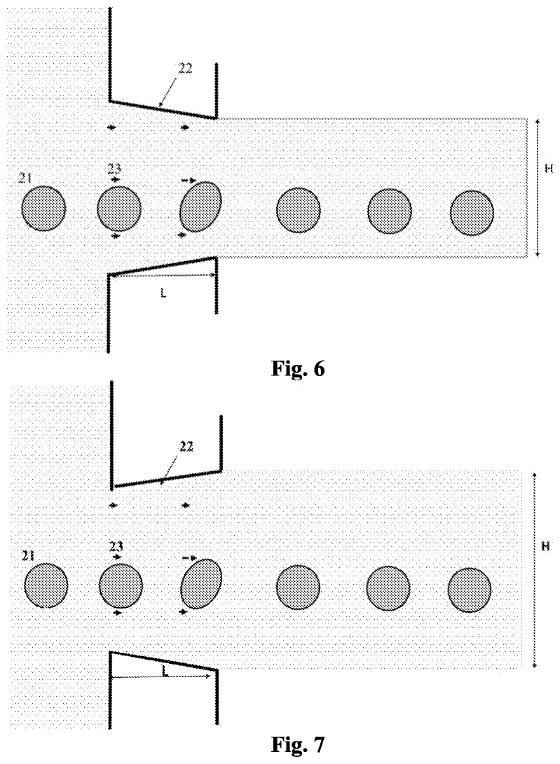

FIG. 5 illustrates the travelling of a vesicle or a liposome (21) through an orifice with length L and diameter H. Before the entrance of the orifice the vesicle (21) is round due to the absence of a large shear flow. When the vesicle passes the orifice, it will be subject to a shear flow (23), becomes elongated, but will not break. The vesicle next enters in the jet part and will attain again its spherical shape.

FIG. 6 illustrates the travelling of a vesicle or a liposome (21) through a positively tapered orifice with length L and diameter H, exhibiting plug flow. Before the entrance of the tapered orifice the vesicle is round and due to the small shear in the plug flow it remains mainly spherical. The vesicle subsequently enters in the jet part and attains its spherical shape.

FIG. 7 illustrates the travelling of a vesicle or a liposome (21) through a negatively tapered orifice with length L and diameter H, also known as a diffuser. Due to the widening, there is a reduction of shear on the nanoparticles in the flow.

It will be clear that tapering orifices with a wide tapering angle according to the invention may also be chosen to have a total length substantially larger than the most narrow diameter, because the detrimental effect on the integrity of the nanoparticles is the largest in the most narrow part of the tapering orifice with a narrow part length L to narrow diameter H ratio as presented according to the invention.

Vesicles and liposomes are also frequently used in gene therapy, in particular to promote the delivery of encapsulated RNA and DNA vectors to the lungs. These vesicles can typically range in size between 0.02 and 2 micron, making them potentially susceptible to shear-induced breakage during aerosolization. In Table 3 below some results are summarized on shear degradation of a number of these vesicles.

TABLE 3

| | | Breakage (%) | | | |
|---|---|---|---|---|---|
| | | Vibrating | Breakage (%) | Breakage (%) | Breakage (%) |
| Vesicle size | Liposome | Membrane | t = 200 ns | t = 50 ns | t = 10 ns |
| nanometre | (molar ratio) | Nebulizer | Orifice 2 μm | Orifice 2 μm | Orifice 2 μm |
| 44 | HSPC:CH (1:1) | 5 | 0 | 0 | <1 |
| 78 | HSPC:CH (1:1) | 14 | 0 | 0 | <3 |
| 126 | HSPC:CH (1:1) | 26 | <1 | <1 | <4 |
| 254 | HSPC:CH (1:1) | 43 | <2 | <1 | <6 |
| 780 | HSPC:CH (1:1) | 38 | <3 | <3 | <8 |
| 1250 | HSPC:CH (1:1) | 64 | <10 | <5 | <10 |
| 500-1200 | Curosurf | >90 | <20 | <20 | >40 |

Using a vibrating membrane nebulizer with a formulation reservoir of 1 ml aerosol droplets in the range of 2-10 micron have been produced during a nebulization time of 8 minutes. All liposome formulations (15 μg/ml) with hydrogenated soy phosphatidylcholine (HSPC) and cholesterol (CH) suffer from breakage, larger vesicles more than smaller ones. According to the invention aerosol droplets with an average size of 4 micron have also been made by passing the same formulations with the vesicles through an orifice having a diameter H=2 micron and a length L=1 micron at an average velocity V=5 m/s. Because of plug flow conditions the volume averaged wall shear rate will be significantly lower than 4V/H=10×10⁶ per second, whereas the passage time t is about 100 nanosecond. Due to this short shearing time according to the invention the vesicles present in the droplets hardly suffer from degradation or breakage. At an average velocity of V=20 m/s the passage time t through an untapered orifice is about 50 nanosecond and less degradation is found than in the case of an average velocity of 10 m/s. Increasing the average velocity to V=100 m/s (t=10 ns) however leads to a substantial degradation of the formulation. Decreasing the nozzle length L to 0.5 micron (H=2 micron), gives a degradation loss that is relatively 10-40% less than in the case of a 1 micron length nozzle. A major relative reduction in breakage was found for the pulmonary surfactant Curosurf in comparison with the vibrating membrane nebulizer. Results with 2 micron nozzles having a hydrophobic fluor coating with a water contact angle of 112° yield also a smaller degradation loss typically of relatively 5-20% less.

With preference a formulation is provided that comprises lipid nanoparticles (LNP) or hybrid nanoparticles with polymers (HNP) with a length or size A larger than 20 nanometre and which LNP's or HNP's having a content comprising other nanoparticles taken from a group, containing proteins, biological molecules, DNA, RNA, mRNA and antibodies. With this method the large nanoparticles vulnerable to shear degradation are more safely protected.

It will be clear that tapering orifices with a wide tapering angle according to the invention may also be chosen to have a total length substantially larger than the most narrow diameter, because the detrimental effect on the integrity of the nanoparticles is the largest in the most narrow part of the tapering orifice with a narrow part length L to narrow diameter H ratio as presented according to the invention.

1. Method for delivering nanoparticles in an atmosphere, comprising:
providing nano-particles having a particle length (λ) in a liquid to form a liquid formulation;
pressurizing said liquid formulation to a moderate operating pressure (p) to provide a pressurized liquid formulation; and
feeding said pressurized liquid formulation through a spray nozzle orifice, having a channel length (L) between an inlet and an outlet of said orifice and an average channel diameter (H) between said inlet and said outlet, to create a liquid stream of said liquid formulation with a velocity;
wherein said moderate operating pressure is held below 10 MPa and said velocity less than 100 m/s;
wherein said orifice has a channel length (L) that is shorter than said average channel diameter (H); and
wherein said liquid stream is collected at said outlet as a jet of consecutive liquid droplets and wherein each contain at least one nanoparticle of said nanoparticles.

2. Method according to claim 1, wherein said liquid formulation comprises shear stress-sensitive nanoparticles taken from a group, containing complex proteins, large biological molecules, long chain DNA & RNA, viruses, large vesicles, liposomes, bacteriophages, and antibodies; and
wherein said orifice has a channel length (L) that is shorter than half said average channel diameter (H).

3. Method according to claim 2, wherein said orifice has a channel length (L) that is at most a quarter of said average channel diameter (H).

4. Method according to claim 2, wherein said liquid formulation comprises protein and/or antibody molecules, and/or nucleotide compounds like DNA or RNA molecules, with a molecular weight that is larger than 100.000 g/mol.

5. Method according to claim 2, wherein said liquid formulation comprises bacteriophages with an average size larger than 20 nanometre.

6. Method according to claim 2, wherein said liquid formulation comprises lipid nanoparticles or liposomes, in particular lung surfactants, of which said length λ is larger than 20 nanometre.

7. Method according to claim 6, wherein said liquid formulation comprises vesicles that have a content comprising nanoparticles taken from a group, containing proteins, biological molecules, DNA, RNA, vaccines, viruses, bacteriophages and antibodies with a molecular weight above 100.000 Da.

8. Method according to claim 2, wherein said nozzle orifice has a substantially constant diameter (H) that is between 1 micron and 10 micron.

9. Method according to claim 2, wherein said nozzle orifice has an average diameter (H) between 1 micron and 10 micron; and wherein said orifice tapers over at least part of said length from said inlet to said outlet.

10. Method according claim 9, wherein said nozzle orifice is provided with a positive taper, narrowing from said inlet entrance to said outlet at substantially a tapering between 5° and 45°.

11. Method according to claim 1, wherein an inner wall of said nozzle orifice is provided with a hydrophobic slip flow enabling coating.

12. Method according to claim 1, wherein a product of a mass density (ρ) of said fluid, a fluid velocity (V) inside said orifice and said nozzle diameter (H) divided by a viscosity (η) of said fluid, expressed as ρ·V·H/η, is maintained below 2.500.

13. Method according to claim 1, wherein said nanoparticles have a maximum particle length λₘₐₓ before breakage upon elongation; wherein said liquid formulation is subjected to a wall shear rate γ_{wall} [per second] while passing through said spray nozzle orifice; and wherein said liquid formulation is exposed within said spray nozzle orifice to said wall shear rate during a shear time (t) that is less than λₘₐₓ/(λ·γ_{wall}) seconds.

14. Method according to claim 13, wherein said wall shear rate γ_{wall} is well above 100.000, in particular above 1.000.000 per second.

15. Method according to claim 1, wherein said nano-particles comprises macromolecules with a molecular weight that is larger than 100.000 g/mol; and wherein said macromolecules have a ratio λₘₐₓ/λ of at least 2, and preferably a ratio λₘₐₓ/λ of at least 4.

16. Method according to claim 1, wherein said nozzle orifice is part of a collection of substantially identical nozzle orifices that extend through a common membrane layer that is supported by a substrate, wherein said substrate has at least one cavity extending to said nozzle orifices of said collection of orifices, and wherein said liquid formulation is delivered at said operating pressure jointly to said cavities to supply said nozzle orifices of said collection of orifices.

17. Method according to claim 3, wherein said liquid formulation comprises protein and/or antibody molecules, and/or nucleotide compounds like DNA or RNA molecules, with a molecular weight that is larger than 100.000 g/mol.

18. Method according to claim 3, wherein said liquid formulation comprises bacteriophages with an average size larger than 20 nanometre.

19. Method according to claim 3 wherein said liquid formulation comprises lipid nanoparticles or liposomes, in particular lung surfactants, of which said length 2 is larger than 20 nanometre.

20. Method according to claim 3, wherein said nozzle orifice has a substantially constant diameter (H) that is between 1 micron and 10 micron.
